(19) United States
(12) Patent Application Publication
(10) Pub. No.: US 2010/0143270 A1
(43) Pub. Date: Jun. 10, 2010

(54) THERAPEUTIC COTININE COMPOSITIONS

(75) Inventors: David Albert Scott, Louisville, KY (US); Michael Martin, Louisville, KY (US)

Correspondence Address: VIKSNINS HARRIS & PADYS PLLP, P.O. BOX 111098, ST. PAUL, MN, 55111-1098, US

(73) Assignee: University of Louisville Research Foubdation, Louisville, KY (US)

(21) Appl. No.: 12/528,050

(22) PCT Filed: Feb. 21, 2008
(86) PCT No.: PCT/US08/54569

§ 371 Date: Mar. 1, 2010

Related U.S. Application Data
(60) Provisional application No. 60/890,954, filed on Feb. 21, 2007.

(51) Int. Cl. A61K 31/4439; A61P 29/00; A61K 9/68; A61P 19/02; A61P 35/00

(52) U.S. Cl. 424/48; 514/343

(57) ABSTRACT

The present invention provides methods and compositions for treating acute and/or chronic inflammation with cotinine or a pharmaceutically acceptable salt thereof.

### CLAIM OF PRIORITY

This patent application claims priority to U.S. Application Ser. No. 60/890,954 filed on Feb. 21, 2007. The instant application claims the benefit of the listed application, which is hereby incorporated by reference herein in its entirety.

### BACKGROUND OF THE INVENTION

Inflammation is an important pathologic process. As an indicator of disease, inflammation is directed at destroying harmful agents and initiating repair, and is usually associated with warmth, redness, swelling, and pain caused by a series of cellular and tissue responses to some injurious agent. The response is directed at the inciting agent or at least rendering it harmless. In the process, inflammation also prevents the spread of the inciting injurious agent. All this activity may cause damage or destruction to normal tissue in the immediate area. The inflammatory process cleans up resulting debris and starts restoring tissue to a normal state.

Many different agents can provoke an inflammatory response. For example, a physical agent, such as solar radiation produces inflammation (e.g., sunburn). Chemical agents can cause inflammation (e.g., turpentine). Inflammation is also a component of most hypersensitivity reactions, such as rheumatoid arthritis. Further, biologic agents can cause inflammation, such as the pain, swelling, and pus associated with a bacterial infection.

There are two fundamental types of inflammation: acute and chronic. Acute inflammation presents as a rapid onset, short duration, but can have profound signs and symptoms. Chronic inflammation presents by a slow onset, long duration, and less obvious signs and symptoms. In addition to acute and chronic inflammation, there is also subacute and granulomatous chronic inflammation. Subacute inflammation is an ill-defined form that has some clinical features of acute and some of chronic inflammation. Granulomatous chronic inflammation is a special form of chronic inflammation. This type of inflammation is associated with tuberculosis and some other less common diseases.

Currently, there is a need for compositions and methods for treating acute and/or chronic inflammation. There is also a need for pharmacological tools for the further study of the physiological processes associated with acute and/or chronic inflammation.

### SUMMARY OF THE INVENTION

The inventors have discovered that cotinine [(S)-1-methyl-5-(3-pyridinyl)-2-Pyrrolidinone] suppresses the normal TLR-mediated pro-inflammatory response to pathogens by human monocytes (IL-1α, IL-6, TNF, IL23/IL-12 p40, and IFN-γ); enhances the production of anti-inflammatory molecules (IL-10); and that these phenomena are specifically mediated via the α7 nicotinic acetylcholine receptor (α7 nAChR). Therefore, delivery of cotinine represents an effective therapeutic intervention in acute and/or chronic TLR-mediated inflammatory conditions. Intravenous delivery of cotinine, in addition to cotinine-containing patches, gum, lozenges, inhalers, and enemas, will suppress the pro-inflammatory response of monocytes, enhance the anti-inflammatory response of monocytes, and protect against acute bacterial and viral infections, including naturally occurring sepses; meningitis; post-surgical infections; bioterrorism-introduced diseases; and protect against TLR-mediated inflammatory conditions, including arthritis, bronchitis, some cancers, and vascular inflammation.

The present invention provides methods for treating acute and/or chronic inflammation.

The invention provides a pharmaceutical composition comprising cotinine, or a pharmaceutically acceptable salt thereof, in combination with steroidal and non-steroidal anti-inflammatory drugs (SAIDs and NSAIDs), and a pharmaceutically acceptable diluent or carrier.

Additionally, the invention provides a therapeutic method for preventing or treating an acute and/or chronic inflammatory condition or symptom in a mammal, such as a human, wherein the activity of one or more cytokines is implicated and antagonism or agonism of its action is desired comprising administering to a mammal in need of such therapy, an effective amount of cotinine, or a pharmaceutically acceptable salt thereof.

The invention provides the use of cotinine or a pharmaceutically acceptable salt thereof for the manufacture of a medicament useful for the treatment of anaphylactic shock, arthritis, asthma, cholecystitis, chronic obstructive pulmonary diseases, cystitis, dacryoadenitis, dermatomyositis, dermatitis, endocarditis, endometritis, fasciitis, fibrositis, gastritis, gingivitis, glossitis, mastitis, meningitis, myelitis, myocarditis, nephritis, neuritis, orchitis, osteisis, pancreatitis, parotitis, pericarditis, periodontitis, peritonitis, phlebitis, proctitis, prostatitis, pyelonephritis, rhinitis, sepsis and septic shock, sinusitis, stomatitis, synovitis, tendonitis, tonsillitis, vaginitis, vasculitis, vascular diseases, and/or vulvitis in a mammal, such as a human.

In accordance with the present invention, cotinine or the pharmaceutically acceptable salts thereof, such as the tartrate, aspartate, lactate, malate, citrate, fumarate, sulfate or chloride salts, are used in treating acute and/or chronic inflammation. The present invention provides a therapeutic method of treatment to reduce or alleviate the symptoms of acute and/or chronic inflammation. In one embodiment, the present invention provides a therapeutic method to alleviate or reduce the symptoms of acute and/or chronic inflammation through the administration of any suitable dosage form, e.g., oral administration, injection, sublingual absorption, as a constituent of chewing gum to be chewed by the patient, as a transdermal patch to be applied to the skin of the patient, or through other suitable forms of administration.

The present invention also provides, as an article of manufacture, a packaging material such as a box, bottle, tube, spray or insufflator, intravenous bag, envelope or the like and at least one unit dosage form of a pharmaceutical agent contained in the package wherein the pharmaceutical agent comprises cotinine or a pharmaceutically acceptable salt thereof in an amount effective to alleviate or reduce the symptoms of acute and/or chronic inflammation, and wherein the package includes instructions indicating use for alleviating acute and/or chronic inflammation. Suitable instructions include printed labels, printed package inserts, tags, cassette tapes and the like.

Use of cotinine or a pharmaceutically acceptable salt thereof, in the manufacture of a medicament for the treatment of anaphylactic shock, arthritis, asthma, cholecystitis, chronic obstructive pulmonary diseases, cystitis, dacryoadenitis, dermatomyositis, dermatitis, endocarditis, endometritis, fasciitis, fibrositis, gastritis, gingivitis, glossitis, mastitis, meningitis, myelitis, myocarditis, nephritis, neuritis, orchitis, osteisis, pancreatitis, parotitis, pericarditis, periodontitis, peritonitis, phlebitis, proctitis, prostatitis, pyelonephritis, rhinitis, sepsis and septic shock, sinusitis, stomatitis, synovitis, tendonitis, tonsillitis, vaginitis, vasculitis, vascular diseases, and vulvitis.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter), or both. In still other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

By “modulate” is meant that the inflammatory response is up regulated or down regulated, such that the response is greater than or less than that observed in the absence of the modulator. For example, the term “modulate” can mean “inhibit” but the use of the word “modulate” is not limited to this definition.

By “inhibit,” “down-regulate,” or “reduce,” it is meant that the inflammatory response is reduced below that observed in the absence of the modulator. In one embodiment, inhibition, down-regulation or reduction with cotinine or a pharmaceutically acceptable salt thereof is below that level observed in the presence of a control molecule.

### BRIEF DESCRIPTION OF THE FIGURES

FIGS. 1A-1B. FIG. 1A shows the chemical structure of nicotine, and FIG. 1B shows the chemical structure of cotinine. While nicotine is metabolized by the liver to form several different metabolites, up to 80% of nicotine is converted to cotinine. The first stage in nicotine biotransformation is mediated by a cytochrome P450 system to produce nicotine-Δ^{1′(5′)}-iminium ion, which is in equilibrium with 5′-hydroxynicotine. The second step is catalyzed by a cytoplasmic aldehyde oxidase. Nicotine iminium ion has received considerable interest since it is an alkylating agent and, as such, could play a role in the pharmacology of nicotine.

FIGS. 2A-2B. Human monocytes treated with (FIG. 2A) nicotine or (FIG. 2B) cotinine produce significantly less TNF when stimulated with P. gingivalis (MOI=10). Monocytes were pre-treated for 2 h with nicotine or cotinine stimulated with P. gingivalis for 20 h. Cell-free supernatants were harvested by centrifugation and levels of TNF were determined by ELISA. The tobacco-induced suppression of TNF production is dose-dependent. Note that mean nicotine concentrations in smokers would be expected to be in the region of 30 ng/ml with mean cotinine levels in smokers in the region of 200-500 ng/ml. Systemic cotinine levels of 1-15 ng/ml are found in second-hand smokers. Data are the arithmetic mean±s.d. of 3 experiments. * Indicates statistical significance at p<0.05, as compared to cells stimulated with P. gingivalis alone.

FIGS. 3A-3E. Cotinine inhibits the production of multiple pro-inflammatory cytokines in P. gingivalis-stimulated (MOI=10) human monocytes. Monocytes were pre-treated with cotinine (1000 ng/ml) for 2 hours then stimulated with purified LPS (0 to 1×10⁴ ng/ml) for 24 hours. Cell-free supernatants were harvested by centrifugation and levels of pro-inflammatory cytokines were determined by ELISA. Data are the arithmetic mean±s.d. of 3 experiments. * Indicates statistical significance at p<0.05, as compared to cells stimulated with LPS alone.

FIG. 4. Cotinine increases the release of the anti-inflammatory cytokine IL-10 in LPS-stimulated monocytes. Cells were pre-treated with cotinine (0 to 10,000 ng/ml, respectively) for 2 hours then stimulated with purified LPS for 24 hours. Cell-free supernatants were harvested by centrifugation and levels of IL-10 were determined by ELISA. Data are the arithmetic mean±s.d. of 3 experiments. * Indicates statistical significance at p<0.05, as compared to cells stimulated with LPS alone.

FIG. 5. Alpha-Bungarotoxin treatment of human monocytes abrogates the anti-inflammatory effects of cotinine. Monocytes were pre-treated for 30 minutes with 500 ng/ml of the alpha-7 nACHR antagonist alpha-BTX followed by 2 h with cotinine (1000 ng/ml). Cells were then stimulated with P. gingivalis for 20 h. Cell-free supernatants were harvested by centrifugation and levels of TNF were determined by ELISA. Data are the arithmetic mean±s.d. of 3 experiments. * Indicates statistical significance at P<0.05, as compared to cells stimulated with P. gingivalis alone.

FIGS. 6A-6E. Cotinine dramatically suppressed the production and release of multiple pro-inflammatory cytokines in LPS-stimulated monocytes (FIGS. 7A-7E). Cells were pre-treated with nicotine or cotinine (100 and 1000 ng/ml, respectively) for 2 hours then stimulated with purified LPS (0 to 1×10⁴ ng/ml) for 24 hours. Cell-free supernatants were harvested by centrifugation and levels of pro-inflammatory cytokines were determined by ELISA. FIG. 6A provides data for IFNγ; FIG. 6B provides data for IL-1β, FIG. 6C provides data for IL-6, FIG. 6D provides data for TNF-α, and FIG. 6E provides data for IL-12/IL-23 p40.

FIG. 7. Inhibition of PI3K using LY294002 abrogates the ability of cotinine to suppress the inflammatory response in P. gingivalis-stimulated monocytes. Cells were pre-treated with 25 μM of the PI3K inhibitor LY294002 for 30 minutes followed by 2 h with nicotine or cotinine. Cell-free supernatants were harvested by centrifugation and levels of TNF were determined by ELISA. * indicates statistical significance at P<0.05, as compared to cells stimulated with P. gingivalis alone. Data are the arithmetic mean±s.d. of 3 experiments. All media used in these experiments contained 0.01% DMSO to act the organic solvent control used to solubilize LY294002.

FIG. 8. Human monocytes treated with nicotine or cotinine produce significantly less TNF-α when stimulated with Porphyromonas gingivalis. Monocytes were pre-treated for 2 h with nicotine (triangles) or cotinine (squares) then stimulated with P. gingivalis (MOI=10) for 20 h. Cell-free supernatants were harvested by centrifugation and levels of TNF-α were determined by ELISA. The alkaloid suppression of TNF production is dose-dependent. Mean nicotine concentrations in smokers would be expected to be in the region of 30 ng ml⁻¹ with mean cotinine levels in smokers in the region of 200-500 ng ml⁻¹. Systemic cotinine levels of 1-15 ng ml⁻¹ are found in second-hand smokers. P. gingivalis is a Gram negative periodontal pathogen. Similar results are seen with Escherichia coli and purified LPS (data not shown). Data are the arithmetic mean±s.d. of 3 experiments. * Indicates statistical significance at p<0.05, as compared to cells stimulated with P. gingivalis alone. Cotinine (0 to 10⁵ ng ml⁻¹) had no statistically significant effect on the viability of primary monocytes, compared to P. gingivalis alone.

FIGS. 9A-9E. Cotinine inhibits the production of multiple pro-inflammatory cytokines in P. gingivalis-stimulated human monocytes (A-D) but augments IL-10 production (E). (A-E) Control monocytes (closed squares) and monocytes pre-treated with cotinine (100 ng ml⁻¹) for 2 hours (open squares) were stimulated with P. gingivalis 33277 LPS (0-10⁵ ng ml⁻¹) for 24 hours. Cell-free supernatants were harvested by centrifugation and levels of pro-inflammatory cytokines (A-E) and IL-10 (E) were determined by ELISA. Data are the arithmetic mean±s.d. of 3 experiments. Statistical significance was set at p<0.05, and cotinine altered the production of all cytokines tested, as compared to cells stimulated with LPS alone. Cotinine (100 ng ml⁻¹) had no statistically significant effect on the viability of primary monocytes, compared to LPS alone.

FIG. 10. α-Bungarotoxin treatment of α7AChR-expressing human monocytes abrogates the anti-inflammatory effects of cotinine. Control monocytes (open squares) and monocytes pre-treated for 30 minutes with the α7 nACHR antagonist α-BTX (2 μml⁻¹, closed squares) were incubated for 2 h with various concentrations of cotinine. Cells were then stimulated with P. gingivalis (MOI=10) for 20 h. Cell-free supernatants were harvested by centrifugation and levels of TNF were determined by ELISA. Data are the arithmetic mean±s.d. of 3 experiments. * Indicates statistical significance at p<0.05, as compared to control cells.

FIG. 11. The anti-inflammatory potential of cotinine is a GSK-3β-dependent but NF-κB-independent phenomenon. THP-1 Blue cells are stably transfected with a reporter plasmid expressing secreted embryonic alkaline phosphatase (SEAP) gene under the control of a NF-κB-inducible promoter. THP-1 and THP-1 Blue cells were pre-treated with cotinine (100 ng ml⁻¹) for 2 hours then stimulated with LPS (1 μg ml⁻¹) for 24 hours. Cell-free supernatants were harvested by centrifugation and relative expression levels of SEAP (reflecting NF-κB; THP-1 Blue; circles) and IL-10 (THP-1; bars) were determined by spectrophotometric analysis of SEAP activity and ELISA, respectively. Here we also show that the GSK-3β inhibitor —SB216763-enhances IL-10 production in response to LPS, but it is less potent than even low doses of cotinine (10 ng ml⁻¹; p<0.05). Induction of NF-κB on stimulation with LPS is not influenced by cotinine treatment. All data are the arithmetic mean±s.d. of 3 experiments. *p<0.05; compared to unstimulated cells. Cotinine (100 ng ml⁻¹) had no statistically significant effect on the viability of THP-1 or THP-1 Blue cells compared to LPS alone.

FIGS. 12A and 12B. Innate immune suppression by cotinine requires the α7 nAChR-dependent enhancement of PI3K activation. (A) Inhibition of PI3K using LY294002 abrogates the anti-inflammatory effects of cotinine. Cells were pre-treated with (closed triangles) and without (closed squares) 25 μM of the PI3K inhibitor LY294002 for 30 minutes followed by 2 h with various concentrations of cotinine. Following 24 hr P. gingivalis stimulation (MOI=10), cell-free supernatants were harvested by centrifugation and levels of TNF-α were determined by ELISA. * Indicates statistical significance at p<0.05, as compared to cells stimulated with P. gingivalis alone (no LY294002). Data are the arithmetic mean±s.d. of 3 experiments. All media used in these experiments contained 0.01% DMSO to act the organic solvent control used to solubilize LY294002. Similar results are also seen with the alternative PI3K inhibitor, wortmannin (100 nM; data not shown).

(B) Cotinine (or nicotine) treated monocytes exhibit increased PIP3 levels when stimulated with LPS in an α7 nAChR-dependent manner. Cells were stimulated for 60 minutes with 1 μg ml⁻¹ of LPS. Cells were analyzed for PI3K activation by the ability of PI3K to produce PIP3. Activated PI3K produces PIP3 that, in turn, activates downstream signaling components of the PI3K pathway. Levels of PIP3 were determined by ELISA using a competitive inhibition assay (Echelon Bioscience). The specific PI3K inhibitor LY294002 was used at 25 μM (cells were pre-treated for 1 hour) and served as a control to block PI3K activity. α7 nAchR-dependence was established through the use of the α7 nAChR antagonist, α-bungarotoxin. Data are the arithmetic mean±s.d. of 3 experiments.

* Indicates statistical significance at p<0.05, as compared to unstimulated control cells.

¶ Indicates statistical significance at p<0.05, as compared to cells stimulated with LPS alone.

§ Indicates statistical significance at p<0.05 for cells stimulated with LPS, nicotine and LY294002 (or α-BTX) compared to LPS and nicotine only.

† Indicates statistical significance at p<0.05 for cells stimulated with LPS, cotinine and LY294002 (or α-BTX) compared to LPS and cotinine only.

FIG. 13. Enhancement of IL-10 release by cotinine requires the α7 nAChR-dependent enhancement of PI3K activation. Inhibition of PI3K using LY294002 abrogates the anti-inflammatory effects of cotinine and nicotine, as assessed by IL-10 release. Cells were pre-treated with and without the PI3K inhibitor LY294002 (25 μM); and with or without α-bungarotoxin (2 μml⁻¹) for 30 minutes, followed by 2 h with nicotine (100 ng ml⁻¹) or cotinine (100 ng ml⁻¹). Following 24 hr LPS stimulation (2 μml⁻¹), cell-free supernatants were harvested by centrifugation and levels of IL-10 were determined by ELISA. Data are the arithmetic mean±s.d. of 3 experiments.

* Indicates statistical significance at p<0.05, as compared to unstimulated control cells.

¶ Indicates statistical significance at p<0.05, as compared to cells stimulated with LPS alone.

§ Indicates statistical significance at p<0.05 for cells stimulated with LPS, nicotine and LY294002 (or α-BTX) compared to LPS and nicotine only.

† Indicates statistical significance at p<0.05 for cells stimulated with LPS, cotinine and LY294002 (or α-BTX) compared to LPS and cotinine only.

### DETAILED DESCRIPTION

### Acute and Chronic Inflammation

Acute inflammation immediately follows injury to tissues by physical, chemical, or biologic agents. The events following such injury involve blood vessel changes allowing entrance of certain blood leukocytes into the injured area. As these cells grapple with the agent that provoked their appearance, normal surrounding tissue may be damaged or even killed. A series of events involving small blood vessels, blood leukocytes, and tissue cells reacting to an “injurious” agent results in weakening, destruction, and isolation of the agent.

The autonomic nervous system plays a role in inflammation in that blood vessel dilation can be caused by nerve impulses. Arterioles are “hard-wired” to the autonomic nervous system. This means that certain nerve impulses cause contraction of smooth muscle in arteriolar walls while others cause smooth muscle relaxation. Autonomic impulses play a role in relaxation of arteriole smooth muscle so that these vessels can dilate.

Many chemical compounds can cause blood vessel dilation. A host of chemicals have been identified that mediate or otherwise influence a number of responses associated with inflammation. There are four classes of chemical mediators.

The first are of compounds known as “vasoactive amines.” There are two important vasoactive amines, histamine and serotonin, both of which are powerful vasodilators. Histamine is found in mast cells while serotonin is found in blood platelets. Beyond its known vasodilator functions, serotonin's role in inflammation is not clear. Much more is known about histamine. It is well known, for example, that mast cell granules are histamine-filled secretory vesicles which can, when released, produce powerful dilation of blood vessels. If a lot of histamine is released all at once, a life-threatening anaphylactic reaction may ensue. In regular inflammatory responses, however, histamine is released in small amounts in the immediate area of tissue injury. It is in these settings that histamine acts to dilate blood vessels.

The second are a group of proteins constituting the “kinin system.” It is the activation of this system that produces another powerful vasodilator known as “bradykinin.” Initial activation results from exposure of collagen to blood plasma; such exposure is caused by injury to the endothelial blood vessel linings allowing plasma to contact collagen in underlying basement membranes. Following collagen exposure, a series of reactions starting with activation of factor XII leads, ultimately to formation of bradykinin. Blood vessel injury can also lead to blood clot formation by activation of a related system—the blood clotting cascade. Following blood vessel injury, bradykinin causes dilation of small blood vessels in the injured area.

Third, a series of plasma proteins (C1-C9) are activated by the presence of antigenic agents. These plasma proteins constitute the “complement system” or the “complement cascade.” Activated form of at least one of these proteins (C5a) binds on sensitized mast cells causing them to release histamine. Because of the anaphylactic nature the release of large amounts of histamine produces, C5a and other related complement proteins are sometimes known as “anaphylatoxins.”

Finally, the production of “prostaglandins” produces vasodilatation in the area of tissue injury. These are substances produced by a series of reactions from the damaged cell membranes and the subsequent release of “arachidonic acid.” It is arachidonic acid derivatives that become the vasodilator prostaglandins.

Unchecked acute inflammation can lead to death through anaphylactic or septic shock.

The second major form of inflammation is chronic inflammation, which is an immune reaction to some “mild” but persistent antigen that produces a proliferation of lymphocytes (T cells) and/or plasma cells (B cells). This type of inflammation is characterized by an insidious onset and long duration. The signs and symptoms of chronic inflammation are not as dramatic as those associated with acute inflammation. If an inflammatory reaction starts as acute but persists, it will enter a chronic phase.

Such persistence is usually caused by either the inability to eliminate an offending agent, or the continual reacquisition of the offending agent. It also may occur when there is continual exposure to some inanimate materials like pollens and dusts. More often than not, chronic inflammation arises without going through an acute phase first (de novo chronic inflammation). Two examples of this are persistent infections and autoimmune diseases. Microorganisms with low virulence may initiate chronic inflammation. Infection with a microorganism of low virulence that cannot be eliminated easily may result in chronic rather than acute inflammation. Tuberculosis and some dental conditions are examples of such infections.

Sometimes a patient may be “allergic” to her/his own cells. This condition is known as autoimmunity. In these cases, the affected patient's cells serve as a source of constant stimulation of the chronic inflammatory process. Systemic lupus erythematosus and rheumatoid arthritis are autoimmune diseases characterized by chronic inflammation.

Many different cells may be involved in chronic inflammation. The mixture of cells associated with chronic inflammation is different than the mixture associated with acute inflammation. In chronic inflammation, macrophages and lymphocytes are the predominant cells; there are generally few, if any, neutrophils.

Macrophages are prominent in chronic inflammatory exudates. Macrophages are monocytes that entered an area of tissue injury. They can live for months and can thrive in acid environments. In order for macrophages to carry out their functions, they must be stimulated (activated) by chemical mediators. Among the chemical mediators are lymphokines (cytokines secreted by lymphocytes), fibronectin-coated surfaces, and mediators that initiate acute inflammation. Macrophages are excellent phagocytes. They engulf and process antigens allowing them to be neutralized by other cells (lymphocytes). Activated macrophages can also engulf and kill certain microorganisms. Macrophages also secrete a number of substances that assist in the recruitment of other cells (monokines) and cause tissue destruction (collagenases, elastases, reactive oxygen).

T-Lymphocytes are the most characteristic cell of chronic inflammation. Lymphocytes emigrate from blood vessels late in an inflammatory reaction. Lymphocytes account for about 33% of the circulating leukocytes. There are two major types of lymphocytes: T and B. T lymphocytes arise from the thymus gland and are responsible for cell-based immunity. B lymphocytes, on the other hand, arise from bone marrow and are responsible for humoral immunity. T cells must be activated before they carry out their functions. Such activation is effected by monokines (secretory stimulants from monocytes [macrophages]) and, in some cases, directly by antigens. Once activated, lymphocytes can react with certain antigens destroying them or rendering them harmless. They also secrete lymphokines that stimulate macrophages. Thus, macrophages and lymphocytes are interdependent—the activation of one stimulates the activation of the other.

B-lymphocytes (plasma cells) are derived from activation of a class of lymphocytes known as “B cells.” They do not circulate in the blood stream but are transformed in lymphoid organs or at the site of chronic inflammation. They are recognized by their off-center nuclei, abundant basophilic cytoplasms, pale spots near the nuclei (negative Golgi images), and clock-face distribution of nuclear chromatin. Plasma cells manufacture and secrete antibodies against specific antigens. The antibodies that circulate in blood plasma are derived from plasma cells; these circulating antibodies are called “humoral antibodies.” A single plasma cell can only produce antibodies against a single antigen. Once a B lymphocyte is activated, it proliferates creating a clone of cells capable of producing antibodies against the antigen that stimulated it.

Eosinophils are related to neutrophils in that they both display a segmented nucleus. Eosinophils comprise about 3% of the circulating white blood cells and are recognized by the bright red granules within their cytoplasm. These granules are filled with a substance called “major basic protein” that can destroy some parasites and some cells. These cells are not seen in all chronic inflammatory reactions. Rather, they appear in parasitic infestations, hypersensitivity reactions, and some autoimmune conditions.

Another type of cell seen associated with inflammation are multinucleated giant cells, which respond to foreign bodies and certain bacteria. These are huge cells with many nuclei may appear in chronic inflammatory reactions. These cells are formed from the fusion of several macrophages and are called “multinucleated giant cells.” They are often seen associated with foreign particulate matter (splinters, talc, debris). They may also accompany reactions to certain microorganisms of low virulence (e.g., tuberculosis).

Fibroblasts and the collagen they produce are prominent features of chronic inflammation. In fact, over-production of collagen formation may permanently deform inflamed tissues, which is called “fibrosis.” Collagen-producing cells are stimulated by chemical mediators, namely lymphokines and monokines. Once in the area, they produce collagen to replace that which has been destroyed. In the inflammatory reaction does not resolve in a reasonable time, the collagen can build up to scar tissue proportions (fibrosis).

This invention provides a new and useful medicinal treatment used in treating the symptoms of acute and/or chronic inflammation. The treatment concerns the administration to a mammal, and especially a human being, in a pharmaceutically acceptable dosage form, a therapeutically effective amount of cotinine or a pharmacologically acceptable salt thereof for treating the symptoms of acute and/or chronic inflammation. As used herein, the term “treat” means alleviating, reducing symptoms, preventing the onset of symptoms, and/or ameliorating symptoms after onset.

Cotinine is unlike nicotine in key physiological aspects. Cotinine does not appear to upregulate the expression of nicotinic receptors in the brain. It may not be vasoactive, as monitored by changes in heart rate, blood pressure, or skin temperature in response to cotinine infusions in humans. It is considered non-addictive. Also, the administration of cotinine to humans at levels as high as 10 times that attained from cigarette smoking has been shown to be safe.

Tobacco smoke appears to affect susceptibility to and the severity of various skin and mucosal diseases differently. For example, tobacco smoking is associated with an increased incidence and clinical severity of psoriasis and Crohn's disease, but is associated with a lower incidence of pouchitis and celiac disease, and a lower incidence and improved symptoms of ulcerative colitis.

Cotinine Dosage Forms

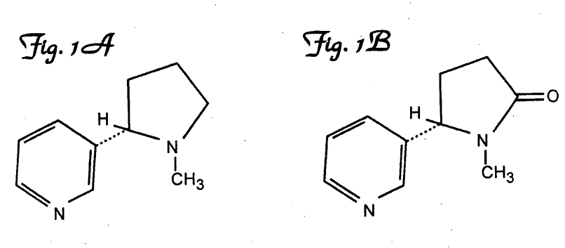

The physiologically active form of cotinine (1-methyl-5-(3-pyridinyl)-2-pyrrolidionone) (FIG. 1B) is the (−)-isomer. As used herein, the term “cotinine” includes (−)-cotinine, or the racemic form, (±)-cotinine. The free base can be employed in the practice of the invention, as can the pharmaceutically acceptable salts. These include the amine-acid addition salts of nontoxic organic acids or inorganic acids, such as the tartarate, fumarate (“scotine”), citrate, maleate, malate, hydrobromide, hydrochloride, sulfate, phosphate and the like. For example, see F. Vaitekunas, J. Amer. Chem. Soc. 79:149 (1957). E. R. Bowman et al. in J. Pharmacol. and Exp. Ther. 135:306 (1962) report the preparation of (−)-cotinine free base from (−)-nicotine. The preparation and purification of (−)-cotinine fumarate is described by N. L. Benowitz et al., Clin. Pharmacol. Ther. 34:604 (1983).

Cotinine is the major metabolite of nicotine which accumulates in the body as a result of nicotine exposure. In contrast to nicotine, cotinine has a relatively long terminal elimination half-life (two versus sixteen hours, respectively). Due to this pharmacological characteristic, cotinine has become the principally used objective biochemical marker of nicotine exposure in cigarette smoking and/or cessation-related research paradigms.

Cotinine is a well-known metabolite of nicotine and is routinely measured in many laboratories.

Pharmaceutically acceptable salts may be obtained using standard procedures well known in the art, for example by reacting a sufficiently basic compound such as an amine with a suitable acid affording a physiologically acceptable anion.

Cotinine can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Cotinine can also be administered in combination with SAIDs and NSAIDs. Examples of such agents include the salicylates, arylalkanoic acids, 2-arylpropionic acids, n-arylanthranilic acids, pyrazolidine derivatives, oxicams, coxibs, and sulphonanilides. Accordingly, in one embodiment the invention also provides a composition comprising cotinine, or a pharmaceutically acceptable salt thereof, at least one other therapeutic agent, and a pharmaceutically acceptable diluent or carrier. The invention also provides a kit comprising cotinine, or a pharmaceutically acceptable salt thereof, at least one other therapeutic agent, packaging material, and instructions for administering cotinine or the pharmaceutically acceptable salt thereof and the other therapeutic agent or agents to an animal to treat anaphylactic shock, arthritis, asthma, cholecystitis, chronic obstructive pulmonary diseases, cystitis, dacryoadenitis, dermatomyositis, dermatitis, endocarditis, endometritis, fasciitis, fibrositis, gastritis, gingivitis, glossitis, mastitis, meningitis, myelitis, myocarditis, nephritis, neuritis, orchitis, osteisis, pancreatitis, parotitis, pericarditis, periodontitis, peritonitis, phlebitis, proctitis, prostatitis, pyelonephritis, rhinitis, sepsis and septic shock, sinusitis, stomatitis, synovitis, tendonitis, tonsillitis, vaginitis, vasculitis, vascular diseases, and vulvitis.

Transdermal devices for the delivery of a wide variety of biologically active agents have been known for some time and representative systems which utilize rate controlling membranes and in-line adhesives are disclosed in U.S. Pat. Nos. 3,598,122; 3,598,123; 3,742,951; 4,031,894, 4,144,317; 4,201,211; 4,379,454; 4,597,961; 4,839,174; 5,230,896; 5,721,257 and 6,165,497, which are incorporated herein by reference. Such devices generally comprise an impermeable backing, a drug or active agent reservoir, a rate controlling membrane and a contact adhesive layer which can be laminated or heat sealed together to produce a transdermal delivery device. Such devices are manufactured to administer cotinine at a pre-determined level, and can be used for short-term or long term delivery. Transdermal cotinine systems can be designed to provide higher steady-state blood levels of cotinine.

Cotinine vapor can be delivered to patients in aerosol form, similar to the inhaler technology used to supply bronchial asthma medications. U.S. Pat. Nos. 5,167,242 and 4,917,120 provide inhaling devices that would allow a user to ingest cotinine vapors orally.

In certain embodiments, cotinine gum is prepared (e.g., U.S. Pat. No. 6,645,470). Cotinine gum is an ion-exchange resin that releases cotinine slowly when the patient chews, and the cotinine present in the mouth is delivered directly to the systemic circulation by buccal absorption. “Buccal administration” refers to any system or device for oral administration of a drug to a patient that is held in the mouth and is used to deliver a drug through the buccal mucosa and into the patient's body. This term includes, but is not limited to, lozenges, capsules, and tablets.

Cotinine can be delivered by means of capsules, tablets, and lozenges. For example, a chewable capsule can be filled with a liquid containing cotinine, together with additives for improving flavor and dispersion (e.g., WO 803803). The capsules are provided in a variety of pH values to allow the patient a choice of cotinine absorption rate. In certain embodiments, a cotinine lozenge is prepared from a mixture of inert filler material, a binder, and either pure cotinine or a cotinine-containing substance by cold compression (e.g., U.S. Pat. No. 4,806,356). In one embodiment a cotinine lozenge is prepared for oral delivery in the form of an inclusion complex of cotinine and a cyclodextrin compound. In certain embodiments, additional flavorings are added, for example, a candy taste, such as chocolate, orange, vanilla, and the like; essential oils such as peppermint, spearmint and the like; or other flavor, such as aniseed, eucalyptus, l-menthol, carvone, anethole and the like.

In certain embodiments a cotinine lozenge is prepared. The cotinine lozenge of the present invention comprises any lozenge, tablet, or capsule formulation that delivers cotinine to the buccal cavity, comprising cotinine dispersed in an absorbent excipient and a nonnutritive sweetener.

According to the compositions and methods described herein, the cotinine is dispersed in an absorbent excipient. Absorbent excipients are pharmaceutically acceptable substances that are capable 1) of reducing the volatility of the cotinine, for example, through absorption or by the incorporation of cotinine, such as in an inclusion complex, and 2) of being compressed into a lozenge or tablet. Suitable absorbent excipients include, but are not limited to, mannitol; cyclodextrins, including α, β, and γ-cyclodextrin, as well as derivatives of cyclodextrins, such as trimethyl-β-cyclodextrin, dimethyl-β-cyclodextrin, hydroxyethyl-β-cyclodextrin, and hydroxypropyl-β-cyclodextrin; silica preparations; cellulosic materials; and other conventional binders and fillers used in the food industry, such as acacia powder, gelatin, gum arabic, and sorbitol. According to some embodiments, the absorbent excipient will serve more than one role in the lozenge formulation. For example, mannitol can function as both a nonnutritive sweetener and an absorbent excipient. Similarly, the absorbent excipient can serve as a flavorant, buffering agent, lubricant, or other component of the lozenge. The absorbent excipient is typically present in an amount between about 5 and 25% by weight (wt %), preferably in an amount between about 5 and 20 wt %, and more preferably in an amount between about 5 and 15 wt %.

In certain embodiments, the lozenge will also contain a nonnutritive sweetener. Typically, a nonnutritive sweetener or combination of sweeteners will be utilized in the lozenges described herein. A nonnutritive sweetener is a synthetic or natural sugar substitute whose sweetness is higher than or comparable to sucrose. Examples of nonnutritive sweeteners include the following: saccharin, invert sugar, cyclamate, palantinose, aspartame, xylitol, acesulfame, sorbitol, monellin, mannitol, neohesperidine, maltitol, palatinit, and sucrose. In a certain embodiments, the nonnutritive sweetener is also noncariogenic. The cariogenicity of a substance is dependent upon its susceptibility to fermentation by Streptococcus mutans and other oral microorganisms. Dental researchers have long recognized that fermentable sweeteners such as sucrose, glucose, starch, and corn syrup are cariogenic or caries causing. The polyol nonnutritive sweeteners, such as xylitol, sorbitol, fructose, invert sugar, palantinose, mannitol, maltitol, palatinit, and ammonium glycyrrhizinate, however, are generally not fermented to any significant degree and are less cariogenic than sucrose. Xylitol has a cooling effect when it dissolves in the mouth, due to its negative heat of solution. Therefore, xylitol is an excellent choice for a sweetener and excipient in a lozenge that needs to be held in the mouth for an extended period of time, and that needs to be taken frequently every day for maximum therapeutic effect. Frequently a combination of nonnutritive sweeteners will be used.

In certain embodiments, the lozenge is a buffered formulation in order to aid in buccal absorption of cotinine. In certain embodiments, the formulation is at a pH of about 6-11 or at a pH of about 7-9. In certain embodiments, the buffered formulations will include sodium carbonate, sodium bicarbonate, sodium phosphate, calcium carbonate, magnesium hydroxide, potassium hydroxide, magnesium carbonate, aluminum hydroxide, and other substances known to those skilled in the art, as well as combinations of the aforementioned substances.

The buffering agent(s) should be present in an amount sufficient to adjust the pH of the lozenge to between 6 and 11, typically, between about 0.1 and 25% by weight (wt %), preferably in an amount between about 0.1 and 10 wt %, and more preferably in an amount between about 0.1 and 5 wt %.

Magnesium stearate and/or hydrogenated vegetable oil may also be added to the formulation as lubricants. Typically, the lubricant will be present in an amount between about 0.1 and 25 wt %, preferably in an amount between about 0.1 and 10 wt %, and more preferably in an amount between about 0.1 and 5 wt %.

The lozenges described herein may also contain a variety of other additives. For example, pharmacologically active ingredients such as sodium monofluorophosphate, sodium fluoride, dextranase, mutanase, hinokitiol, allantoin, aminocaproic acid, tranexamic acid, azulene, vitamin E derivatives, sodium chloride and the like can be added at need. More specifically, since the effects of xylitol and fluoride on dental hygiene are additive, the former can significantly enhance the efficacy of traditional fluoride treatments. Thus, according to one embodiment, fluoride, and more particularly sodium monofluorophosphate or sodium fluoride will be incorporated into a lozenge formulation having xylitol as a nonnutritive sweetener.

In addition, the lozenge may be colored with conventional, pharmaceutically acceptable food coloring agents. Other additives that may be incorporated within the lozenges described herein include, but are not limited to, preservatives, antimicrobial agents, and antioxidants.

Method of Manufacture

The method of manufacture of these lozenges may be any suitable method known in the art, including but not limited to, the addition of a cotinine compound to premanufactured tablets; cold compression of an inert filler, a binder, and either pure cotinine or a cotinine-containing substance (as described in U.S. Pat. No. 4,806,356, herein incorporated by reference); and encapsulation of cotinine or a cotinine compound. See U.S. Pat. No. 5,135,753, herein incorporated by reference, for examples of methods of manufacture of various cotinine lozenges, sublingual tablets, and gelatin capsules.

According to another embodiment, an in situ inclusion complex is created with cotinine and β-cyclodextrin using a kneading technique. Specifically, a small amount of a cotinine-water solution is added to cyclodextrin and kneaded or mixed. This method of forming the cotinine-cyclodextrin inclusion complex minimizes the use of solvents or diluents and thus, eliminates a purification step in the manufacturing process.

A further embodiment of the present invention provides for the production of inclusion complexes of both the cotinine and the flavorant. This embodiment is employed, for example, when an essential oil, or other volatile flavorant, such as carvone or menthol, is used in the lozenge formulation. As in the case of the cotinine inclusion complexes described herein, incorporation of the flavorant into cyclodextrin decreases the volatility of the flavorant and increases formulation stability. In addition, as the flavorant is slowly released from the complex during lozenge administration, the flavorant will “last” longer.

According to this embodiment, a mixture of the cotinine and the flavorant, and optionally water, is added to the cyclodextrin and kneaded. Alternatively, the cotinine inclusion complex and the flavorant inclusion complex can be prepared separately and then mixed prior to lozenge formulation.

According to another embodiment, a portion of the nonnutritive sweetener, preferably xylitol, is utilized to hard coat the cotinine lozenge. Traditional pan coating techniques can be employed. Typically, weight increases of approximately 35% can be accomplished in less than three hours. The lozenges may be packaged in such a manner as to aid in maintaining cotinine stability. Preferred packaging methods include strip lamination in a foil-like material, or packaging in blisters using a Teflon-like material.

The lozenges described herein will typically have a weight of between about 70 and 1000 mg and will contain fairly low doses of cotinine, preferably less than 5 mg, and most preferably from 0.5 to 2.0 mg.

The invention will now be illustrated by the following non-limiting Examples.

### Example 1

### Cotinine is a Potent Suppressor of the Monocytic Pro-Inflammatory Response

Nicotine [(S)-3-(1-methyl-2-pyrrolidinyl)pyridine] is a major component of tobacco and a highly efficient acetylcholine receptor (nAChRs) agonist that triggers the cholinergic anti-inflammatory pathway and subsequently suppresses the release of several pro-inflammatory cytokines from monocytes. However, a multiplicity of side-effects limits the attractiveness of nicotine as an anti-inflammatory therapeutic. Due to structural similarity, the inventors hypothesized that the primary metabolite of nicotine, cotinine [(S)-1-methyl-5-(3-pyridinyl)-2-Pyrrolidinone], is also an anti-inflammatory mediator. The inventors demonstrated that pre-treatment with cotinine dramatically altered the nature of the inflammatory response of monocytes to Gram negative bacteria by abrogating the production of cytokines that are under the transcriptional control of the NF-κB system (TNF, IFN-γ, IL-1, IL-6, IL-12/IL-23 p40) and shifting the response towards an IL-10-dominated anti-inflammatory profile. The inventors also showed that this anti-inflammatory phenomenon is dependent on the monocytic α7 nicotinic acetylcholine receptor (α7 nAChR). Thus, cotinine represents a novel potential anti-inflammatory agent.

Results and Discussion

The ability to regulate against prolonged or excessive inflammation is critical in preventing the onset of septic shock and the host-mediated damage associated with multiple chronic inflammatory diseases. However, the mechanisms that dictate the establishment of pro- versus anti-inflammatory cytokine-dominated environments are poorly understood. In recent years it has become clear that the nicotinic acetylcholine receptor α7 subunit is a critical regulator of inflammation. Acetylcholine, produced by the vagus nerve network, is an endogenous α7 nAChR agonist and an effective suppressor of pro-inflammatory cytokine release by cells of the monocyte/macrophage lineage. Nicotine, an exogenous α7 nAChR agonist, also suppresses the release of TNF, and other pro-inflammatory cytokine, release from activated monocytic cells.

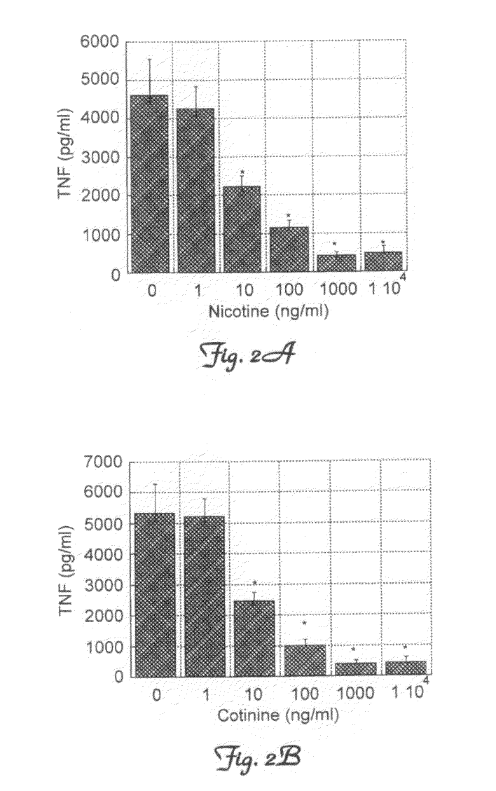

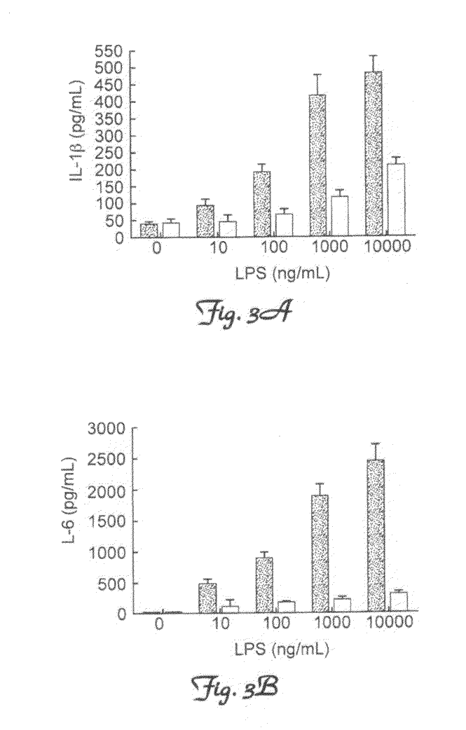

The present results show for the first time that cotinine is a potent anti-inflammatory agent that is active at physiologically relevant concentrations and that cotinine acts specifically via the α7 nACh receptor. As shown in FIG. 2A, nicotine inhibits the release of TNF from P. gingivalis-stimulated monocytes in a dose-dependent manner. Cotinine is equally as effective as nicotine at inhibiting TNF release (FIG. 2B), and blocks >80% of TNF at a dose of 100 ng/ml, a level commonly observed in cigarette smokers.

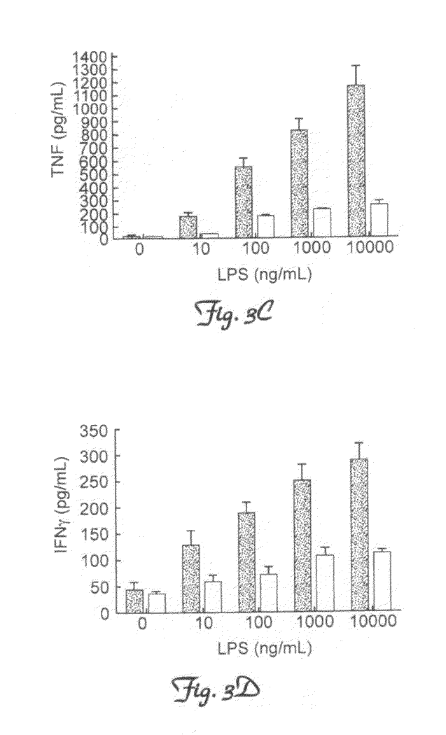

Cotinine inhibited the production and release of multiple pro-inflammatory cytokines by monocytes stimulated with Toll-like receptor (TLR)-agonists in a dose-dependent manner. Cells were pre-treated with cotinine for 2 hours then stimulated with a TLR-agonist (purified LPS, 0 to 1×10⁴ ng ml⁻¹) for 24 hours. Cell-free supernatants were harvested by centrifugation and levels of pro-inflammatory cytokines were determined by ELISA (FIGS. 3A-3E).

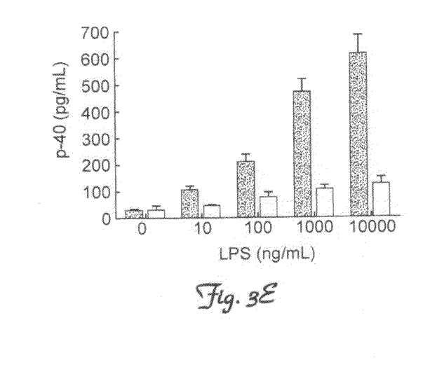

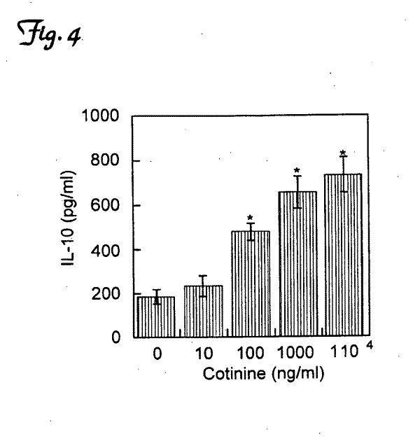

Cotinine increased the production and release of the anti-inflammatory cytokine IL-10 monocytes stimulated with Toll-like receptor (TLR)-agonists in a dose-dependent manner. Cells were pre-treated with cotinine for 2 hours, and then stimulated with a TLR-agonist for 24 hours. Cell-free supernatants were harvested by centrifugation and levels of IL-10 were determined by ELISA (FIG. 4). Data represents the mean (sd) of triplicate experiments.

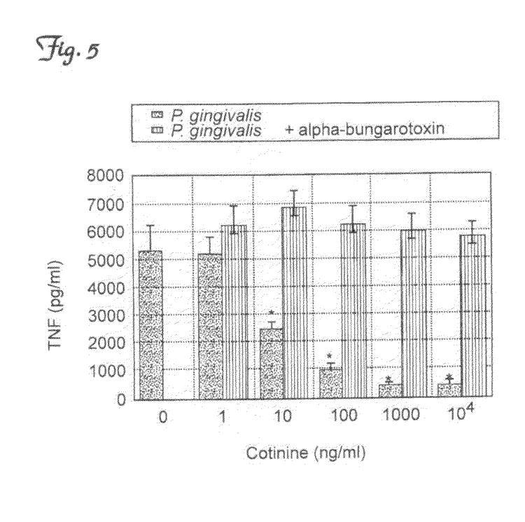

The α7 nACh receptor antagonist, α-BTX, abrogates the anti-inflammatory effects of cotinine in monocytes stimulated with Toll-like receptor (TLR)-agonists. Monocytes were pre-treated for 30 minutes with 500 ng/ml of the α7 nACHR antagonist α-BTX followed by 2 h with cotinine. Cells were then stimulated with a TLR-agonist for 20 h. Cell-free supernatants were harvested by centrifugation and levels of TNF were determined by ELISA (FIG. 5).

Functional AChRs are pentameric and are composed of multiple combinations of a possible 16 monomer subtypes (α1-7; α9-10; β1-4; Δ; ε; and γ) that exhibit divergent pharmacological behaviors. The inventors have used the non-selective nAChR-inhibitor, mecamylamine, to show that cotinine-induced inflammatory suppression is a nAChR-dependent phenomenon. nAChR subtype expression by monocytes and macrophages is highly restricted. Certainly, of the alpha-bungarotoxin sensitive human nAChRs (α1, α7, and α9), monocytes and macrophages express only functional α7 receptors. Therefore, the inventors have been able to use α-bungarotoxin to show that cotinine-induced inflammatory suppression occurs through the nicotinic acetylcholine receptor α7subunit.

The nicotinic anti-inflammatory pathway is unlikely to exist as a single, self-contained entity, rather it must be anticipated that it interacts and converges with multiple other pathways, including the NF-κB pathway and others. For example, the inventors have shown the convergence of the nicotinic anti-inflammatory and an endogenous GSK-3-dependent anti-inflammatory pathway in monocytes. In conclusion, the manipulation of nAChR-initiated signaling pathways likely represents a potentially fruitful area for inflammation research in the coming years and the currently expanding literature suggests that the number of diseases in which the pathway is relevant, for example, pancreatitis, and various vascular pathologies may be expansive.

Materials and Methods

Growth of Porphyromonas gingivalis: P. gingivalis ATCC 33277 was grown in modified gifu anaerobic medium (Nissui Pharmaceutical Co., Tokyo, Japan) under anaerobic conditions (85% N₂, 10% H₂, 5% CO₂) at 37° C.

Isolation and culture of primary monocytes: Whole human blood was purchased from Lampire Biological Laboratories (Pipersville, Pa.). Primary monocytes were isolated by an indirect magnetic monocyte isolation kit (Miltenyi Biotec, Auburn, Calif.). This procedure routinely results in >95% pure CD14⁺ cells, as shown by flow cytometry. Human monocytes were cultured at 37° C. and 5% CO₂ atmosphere, in complete RPMI (RPMI 1640 supplemented with 10% heat-inactivated FBS, 2 mM L-glutamine, 10 mM HEPES, 100 U/ml penicillin G, 100 μg/ml streptomycin, and 0.05 mM 2-mercaptoethanol (Invitrogen™ Life Technologies, Carlsbad, Calif.) plus or minus stimulating agents, as described below.

Inflammatory response of cotinine-exposed monocytes to P. gingivalis and LPS: In an initial series of experiments, we determined how physiological doses of nicotine and cotinine influenced levels of TNF secreted by monocytes stimulated with LPS. Human monocytes were pre-treated for 90 minutes with nicotine (1 to 1,000 ng/ml; Sigma-Aldrich, Inc.) or cotinine (1 to 10,000 ng/ml; Sigma-Aldrich, Inc.), then stimulated with purified E. coli K-12 LPS (0 to 10 μg/ml; InVivoGen™, San Diego, Calif.). TNF concentrations in 24 hr supernatants were measured by ELISA (eBioscience, San Diego, Calif.). Subsequently, the concentrations of multiple cytokines in 24 hr supernatants of cotinine pre-incubated (1000 ng/ml) LPS-(1 μl/ml) stimulated monocytes were measured by ELISA (IFN-γ (PBL Biomed Labs, Piscataway, N.J.), IL-1β, IL-6, TNF, IL-10 (all eBioscience), and IL-12/IL-23 p40 (R&D Systems, Minneapolis, Minn.).

The role of the α7-nicotinic receptor in cotinine-induced inflammatory suppression: Of those human nAChRs sensitive to α-BTX (α1, α7, and α9), monocytes and macrophages express functional α7 receptors only. Therefore, the inventors pre-incubated monocytes with a non-selective nAChR antagonist, mecamylamine (10 μM; Sigma Chemical Co.), and α-BTX (2 μg/ml; Sigma Chemical Co.) to establish that the anti-inflammatory action of cotinine is mediated through nACh receptors and, specifically, the α7 nACh receptor.

Statistical approaches: Statistical significance between groups was evaluated by ANOVA and the Tukey multiple-comparison test using the InStat program (GraphPad Software, San Diego, Calif.). Differences between groups were considered significant at the level of p<0.05.

### Example 2

### Treatment of Inflammation

Important interactions between the nervous system and the inflammatory response have recently been discovered at the molecular level, especially concerning neurophysiological anti-inflammatory mechanisms. A key pathway in communication between the nervous and inflammatory systems is the cholinergic anti-inflammatory pathway, triggered by the interaction of acetylcholine (ACh) with leukocyte nicotinic acetylcholine receptors (nAChRs) resulting in the suppression of the production and/or release of specific pro-inflammatory cytokines. Thus, nAChRs and downstream components of the cholinergic anti-inflammatory pathway present novel therapeutic targets for inflammatory diseases. The present inventors have recently identified an endogenous Toll-like receptor (TLR)-initiated anti-inflammatory pathway in human monocytes that is GSK-dependent. It is believed that the cholinergic and endogenous monocyte GSK-dependent anti-inflammatory pathways may be convergent. These anti-inflammatory pathways hold enormous therapeutic potential.

Lipopolysaccharides (LPS), cell wall components of Gram negative bacteria, are potent inducers of the inflammatory response. LPS recruits, activates, and promotes degranulation events in the most numerous inflammatory leukocyte, the neutrophil. LPS and neutrophil degranulation products each recruit monocytes and macrophages to the locus of infection. While neutrophils are, in relative terms short-lived and transcriptionally quiescent, monocytes/macrophages are longer-lived cells that when stimulated by LPS, and other inflammatory mediators, produce large amounts of pro-inflammatory cytokines, including TNF-α, IL-1, IL-6, IL-12/IL-23 p40, IL-18, and HMGB-1 de novo. These macrophage-derived mediators amplify and direct the inflammatory response and link the innate and adaptive immune responses. In addition to pro-inflammatory cytokine functions of HMGB-1, the continued production of the protein is a requisite for survival in monocytes, with apoptosis occurring when HMGB-1 translation is suppressed.

The ability of the host's immune system to initially recognize and respond to microbes or their components is largely mediated by the innate immune system via the expression of a family of type I transmembrane receptors, the Toll-like receptors (TLRs) which, when activated by bacterial ligands, signal the production of pro-inflammatory cytokines. Most gram negative bacteria, including P. gingivalis, produce monocyte-activating lipopolysaccharides. Lipid A activation of TLR4 on monocytes and macrophages triggers the biosynthesis of diverse mediators of inflammation, such as TNF-α and IL1-β, and activates the production of costimulatory molecules required for the adaptive immune response, including IL-12 p70/IL23 p40 that is critical for the development of IFN-γ producing Th1 cells and influences cell-mediated immunity and IgG2a antibody production from B cells. These inflammatory events are critical in clearing bacterial pathogens locally. However, prolonged local inflammation can result in irreversible tissue damage, and systemic overproduction of pro-inflammatory mediators can result in Gram-negative septic shock which can lead to disseminated intravascular coagulation, multiple organ failure, and death. Inflammatory reactions are critical events in surviving infections, and yet inflammation is a leading cause of morbidity and mortality in humans. TNF-α has been found to be a key mediator of chronic inflammatory diseases, and has been considered a key mediator of septic shock.

The onset of sepsis has been associated with a predominant production of proinflammatory cytokines including IL-1, TNF, IFN-γ and IL-12. Additionally, it has been shown that there is a subsequent set of cytokines, including HMGB1, that play a predominant role in mediating mortality in the latter phase of septic shock. Therefore, the potential of targeted suppression of the inflammatory response is obvious and enormous. In monocytes/macrophages, LPS activates TLR-4 and transduces the activation of multiple intracellular pathways, notably including the NF-κB pathway. The NF-κB pathway is critical in the activation of monocytes/macrophages and in the production of multiple pro-inflammatory cytokines in these cells.

The present inventors performed studies investigating whether cotinine could suppress the P. gingivalis and purified LPS-mediated production of pro-inflammatory mediators in monocytes. The results indicated that not just TNF-α, but other pro-inflammatory mediators were indeed suppressed in monocytes. They observed that cotinine suppressed the LPS-induced pro-inflammatory response in human monocytes. Furthermore, this suppression of innate immunity was α7 nAChR-dependent and entailed signaling through the PI3K-Akt pathway involving GSK-3β.

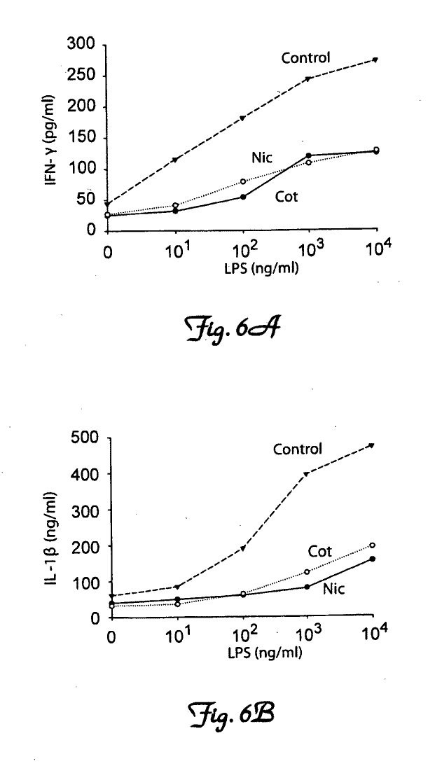

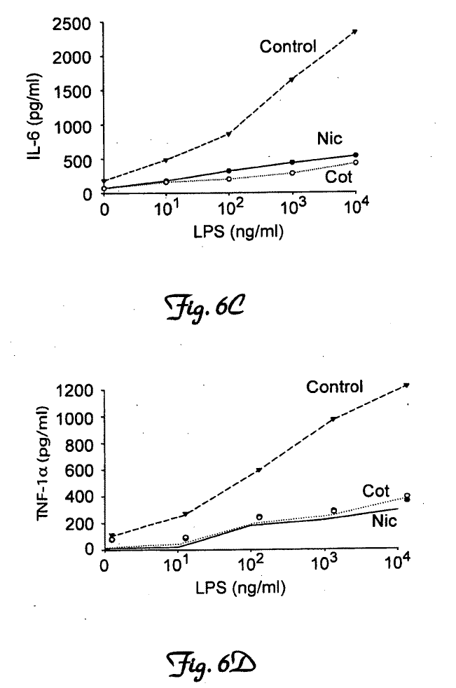

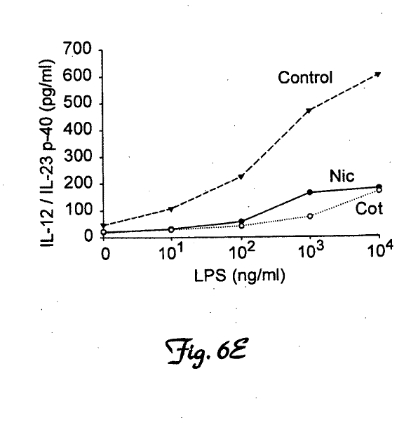

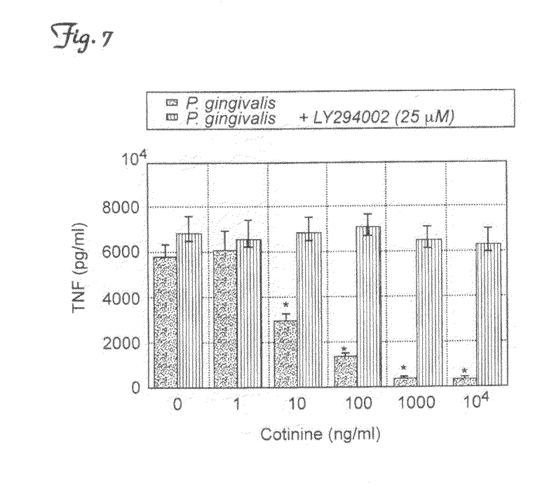

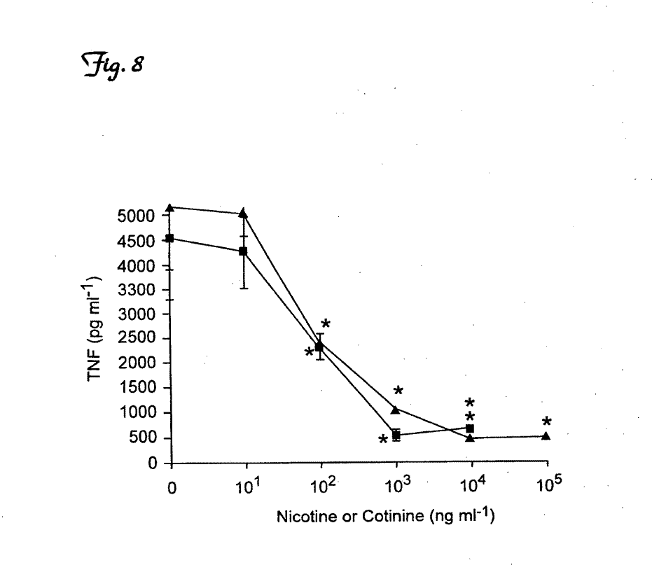

The inventors discovered that cotinine had potent anti-inflammatory properties and that these anti-inflammatory properties are blocked by using a specific α7nACh Receptor antagonist alpha-bungarotoxin (α-BTX). Thus, cotinine is capable of binding to α7nACh receptors on monocytes and inducing anti-inflammatory signaling events. The inventors also observed that human monocytes treated with cotinine produce significantly less TNF-α when stimulated with P. gingivalis (MOI=10). They also observed that α-BTX treatment of human monocytes significantly suppresses the anti-inflammatory effects of cotinine-treated cells when stimulated with P. gingivalis (MOI=10). Cotinine dramatically suppressed the production and release of multiple pro-inflammatory cytokines in LPS-stimulated monocytes (FIGS. 6A-6E). Also, cotinine increased the production and release of the anti-inflammatory cytokine IL-10 in LPS-stimulated monocytes. Inhibition of PI3K using LY294002 abrogates the ability of cotinine to suppress the inflammatory response in P. gingivalis-stimulated monocytes (FIG. 7).

Thus, these studies show that cotinine suppressed the production of pro-inflammatory cytokines, IL-1β, IFN-γ, IL-6, TNF-α, and IL-12/IL-23 p40, but enhanced production of the anti-inflammatory cytokine, IL-10 in LPS-stimulated primary human monocytes. These novel data show for the first time that the primary nicotine metabolite cotinine suppressed the production of multiple pro-inflammatory cytokines in monocytes in response to Gram-negative bacteria.

### Example 3

### Cotinine-Induced Convergence of the Cholinergic and PI3 Kinase-Dependent Anti-Inflammatory Pathways in Innate Immune Cells

The ability to regulate against prolonged or excessive inflammation is critical in preventing the onset of septic shock and the host-mediated damage associated with multiple chronic inflammatory diseases. However, the mechanisms that dictate the establishment of pro- versus anti-inflammatory cytokine-dominated environments are poorly understood. In recent years it has become clear that the nicotinic acetylcholine receptor α7 subunit is a critical regulator of inflammation. Acetylcholine, produced by the vagus nerve network, is an endogenous α7 nAChR agonist. Acetylcholine and the exogenous α7 nAChR agonist, nicotine, both suppress the release of TNF-α from activated cells of monocytic lineage and are protective against LPS-mediated toxicity. Thus, nAChRs and downstream components of the cholinergic anti-inflammatory pathway present novel therapeutic targets for controlling inflammatory diseases.

However, the mechanisms of action of acetylcholine and nicotine in innate immune suppression are still largely unknown. Furthermore, nicotine is rapidly converted into multiple metabolites in humans. While the pharmacological properties of nicotine have been extensively studied, the major proximate metabolite, namely cotinine, has received relatively little attention. Yet, cotinine is a much more stable molecule and systemic concentrations approach ten-fold that of nicotine.

Like nicotine, cotinine evokes striatal dopamine release, appears to improve working memory and attention, is neuroprotective, and suppresses the release of free radicals from neutrophils. Thus, cotinine is clearly pharmacologically active. However, cotinine is unlike nicotine in key physiological aspects. Cotinine does not appear to upregulate the expression of nicotinic receptors in the brain; it may not be vasoactive, as monitored by changes in heart rate, blood pressure, or skin temperature in response to cotinine infusions in humans; it is considered non-addictive; and the administration of cotinine to humans at levels as high as 10 times that attained from cigarette smoking has been shown to be safe.

The present inventors have discovered that cotinine (1-methyl-5-[3-pyridynl]-2-pyrrolidinone) has anti-inflammatory properties.

Materials and Methods:

Isolation and Culture of Primary Monocytes

Whole human blood was purchased from Lampire Biological Laboratories (Pipersville, Pa.). Primary monocytes were isolated by an indirect magnetic monocyte isolation kit (Miltenyi Biotec, Auburn, Calif.) as we have previously reported (M. Martin et al., Nat Immunol. 6 (2005) 777-784; M. Martin et al., J. Immunol. 171 (2003) 717-725). This procedure routinely results in >95% pure CD14⁺ cells, as shown by flow cytometry. Human monocytes were cultured at 37° C. and 5% CO₂ atmosphere, in complete RPMI (RPMI 1640 supplemented with 10% heat-inactivated FBS, 2 mM L-glutamine, 10 mM HEPES, 100 U/ml penicillin G, 100 μg/ml streptomycin, and 0.05 mM 2-mercaptoethanol (Invitrogen™ Life Technologies, Carlsbad, Calif.) plus or minus stimulating agents, as described below.

Growth of Porphyromonas gingivalis

P. gingivalis ATCC 33277 was grown in modified gifu anaerobic medium (Nissui Pharmaceutical Company, Tokyo, Japan) under anaerobic conditions (85% N₂, 10% H₂, 5% CO₂) at 37° C.

Cytokine Release by Monocytes

Mean nicotine concentrations in smokers are in the region of 30 ng with mean cotinine levels in smokers in the region of 200-500 ng ml⁻¹. Systemic cotinine levels of 1-15 ng ml⁻¹ are found in second-hand smokers. Primary monocytes were pre-treated with nicotine (0-100 ng ml⁻¹) or cotinine (0-1000 ng ml⁻¹) then stimulated with the Gram negative periodontal pathogen P. gingivalis (MOI=0-10); purified E. coli K-12 LPS (0-1 μml⁻¹; InVivoGen™, San Diego, Calif.); or purified P. gingivalis 33277 LPS for 20 h. Primary monocytes were also pre-treated with cotinine (100 ng ml⁻¹) then stimulated with increasing doses of LPS for 20 h. Cell-free supernatants were harvested by centrifugation and cytokine levels (TNF, IL-6, IL-10, IL-12/IL-23 p40, as appropriate) were determined by ELISA (eBioscience, San Diego, Calif. or R&D Systems, Minneapolis, Minn.).

Expression of α7 nACh Receptors

Total primary monocyte cell lysate (40 μg of protein) western blots were probed with an anti-α7AChR specific antibody (Q4A163R, BIODESIGN International®, Saco, ME) recognizing amino acids 493-502 at the C terminus. Promyelocytic HL-60 cells served as a positive control. Proteins recognized by the anti-α7AChR specific antibody were detected by enhanced chemiluminescence (Amersham Biosciences, Piscataway, N.J.).

NF-κB in Cotinine-Induced Inflammatory Suppression

THP-1 Blue cells are monocyte-like cells that have been stably transfected with a reporter plasmid expressing secreted embryonic alkaline phosphatase (SEAP) gene under the control of a NF-κB-inducible promoter. THP-1 and THP-1 Blue cells (InVivoGen™, San Diego, Calif.) were pre-treated with cotinine (100 ng ml⁻¹) for 2 hours then stimulated with E. coli LPS (1 μg ml⁻¹) for 24 hours. Cell-free supernatants were harvested by centrifugation and relative expression levels of NF-κB and IL-10 were determined by spectrophotometric analysis of SEAP activity and ELISA, respectively.

The Cholinergic Anti-Inflammatory Pathway in Cotinine-Induced Inflammatory Suppression

The importance of α7 nAChR in cotinine-induced modulation of cytokine release profiles was assessed using the selective nAChR antagonist, α-bungarotoxin (α-BTX; 2 μg ml⁻¹; Sigma Chemical Company, St. Louis, Mo.). Of the α-BTX-sensitive nAChRs, monocytes express only α7 nAChR.

The PI3K-Dependent Anti-Inflammatory Pathway in Cotinine-Induced Inflammatory Suppression

Levels of phosphorylated Akt and phoshorylated GSK-3β following cotinine (0-100 ng ml⁻¹) and/or E. coli LPS (1 μg ml⁻¹) treatment were determined by western blot using whole-cell lysates (20 μg) with probing for Akt using a phospho-specific Akt (Ser473) antibody; probing for GSK-3β using a phospho-specific GSK-3β (Ser9; denoted pGSK-3β) antibody; with the blots stripped and re-probed for total p38 to ensure equivalent loading. Antibodies were purchased from Cell Signaling, Beverly, Mass. Blots were visualized by enhanced chemiluminescence. The importance of the PI3K-dependent anti-inflammatory pathway in cotinine-induced inflammatory suppression was also established by pharmacological inhibition of PI3K and GSK-3β using the PI3K antagonists LY294002 (25 μM; Calbiochem®, San Diego, Calif.) and wortmannin (100 nM, Calbiochem); and the GSK-3β inhibitor SB216763 (6 μM; Sigma). Activated PI3K produces PIP3 that, in turn, activates downstream signaling components of the PI3K pathway. Therefore, cells were also analyzed for cotinine-induced PI3K activation by, assaying the ability of PI3K to produce PIP3. Levels of PIP3 were determined by ELISA using a competitive inhibition assay (Echelon Bioscience, Salt Lake City, Utah). The specific PI3K inhibitor LY294002 was used at 25 μM and served as a control to block PI3K activity.

Convergence of the Cholinergic and PI3 Kinase-Dependent Anti-Inflammatory Pathways During Cotinine-Induced Inflammatory Suppression

Interactions between the cholinergic and PI3 kinase-dependent anti-inflammatory pathways were examined by assessing the efficacy of α-BTX (2 μg ml⁻¹) to block activation of the PI3K pathway. PIP3 levels were monitored by ELISA following 60 minutes with LPS (1 μg ml⁻¹) in the presence and absence of cotinine (0-100 ng ml⁻¹).

Statistical Approaches

Statistical significance between groups was evaluated by ANOVA and the Tukey multiple-comparison test using the InStat program (GraphPad Software, San Diego, Calif.). Differences between groups were considered significant at the level of p<0.05.

Results

Cotinine Inhibits TNF Release

Cotinine is equally as effective as nicotine at inhibiting TNF release (FIG. 8). Indeed cotinine blocks more than 80% of bacteria-induced TNF release from monocytes at a dose of 100 ng ml⁻¹ (FIG. 8), a level commonly observed in cigarette smokers.

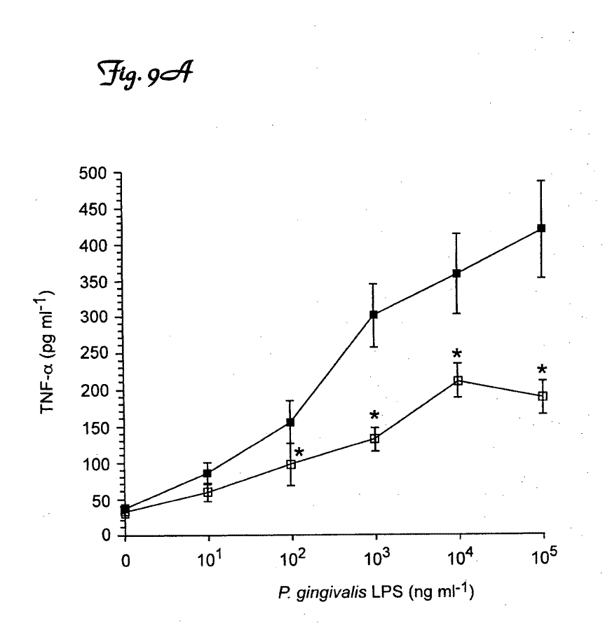

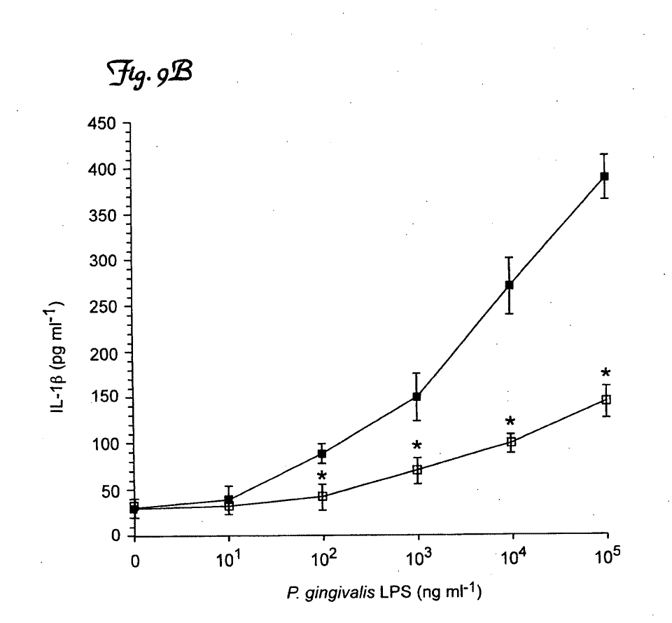

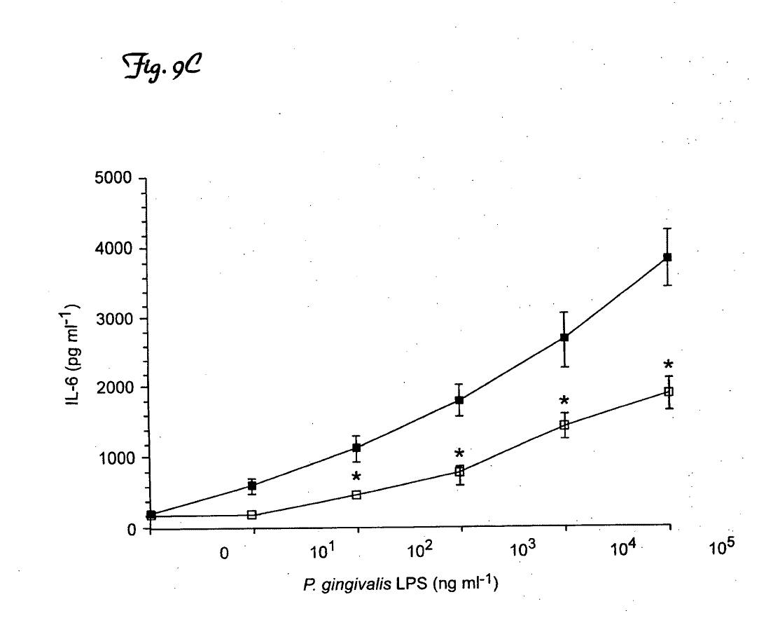

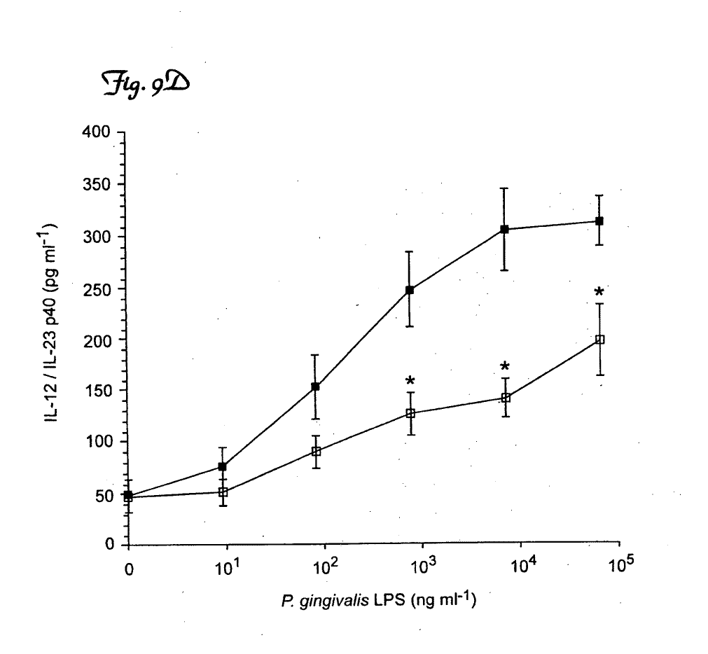

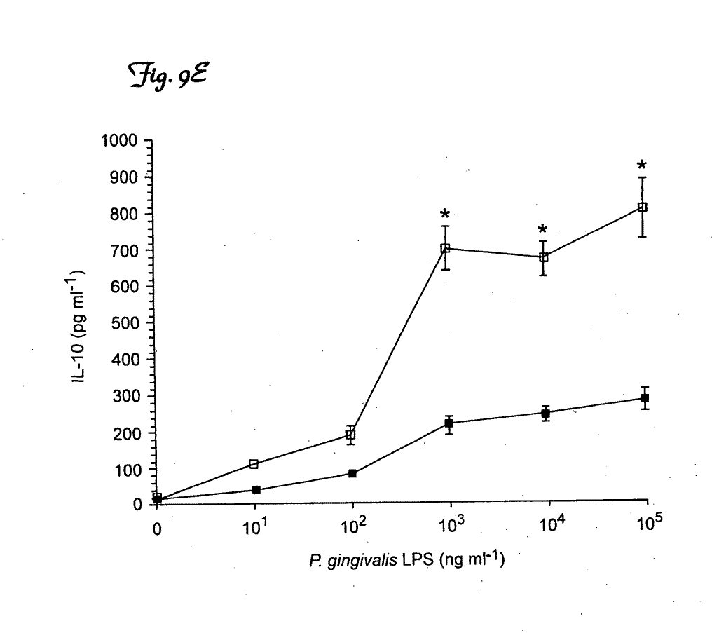

Cotinine promotes an anti-inflammatory phenotype Cotinine also dramatically suppresses the release of multiple pro-inflammatory cytokines (TNF-α, IL-1β, IL-6, and the common IL-12/IL-23 p40 subunit) from monocytes (FIGS. 9A-D) while simultaneously enhancing production of the key anti-inflammatory cytokine, IL-10 (FIG. 9E).

Cotinine-Induced Inflammatory Suppression Likely Occurs Via α7 nAChR

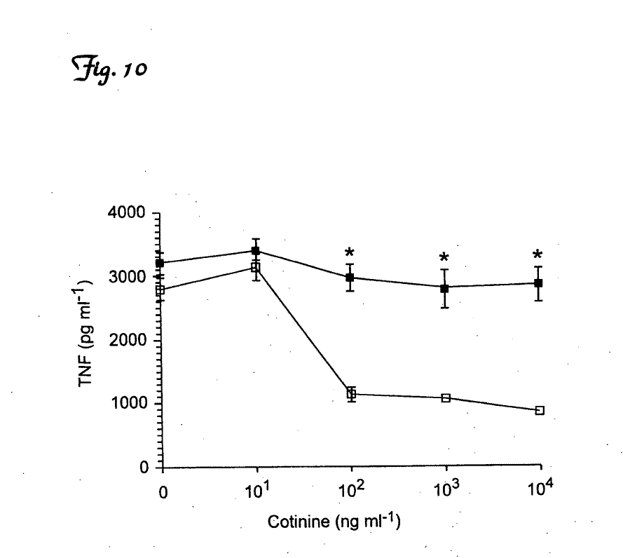

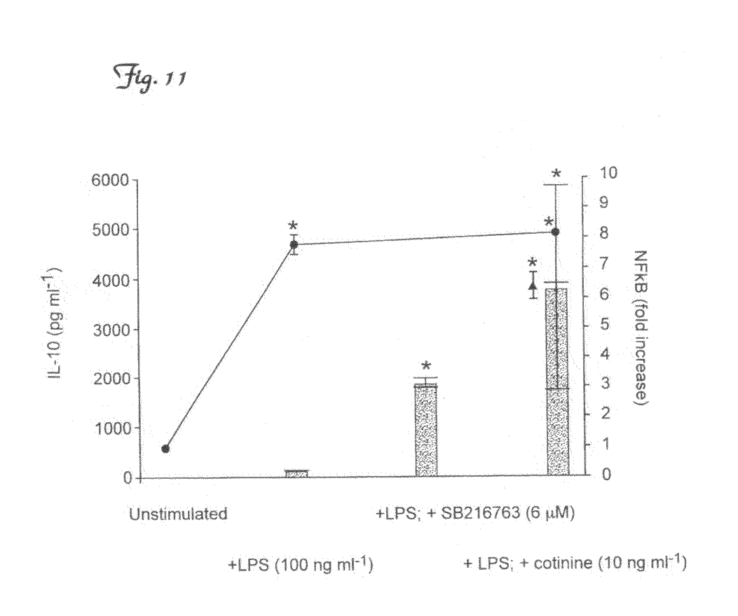

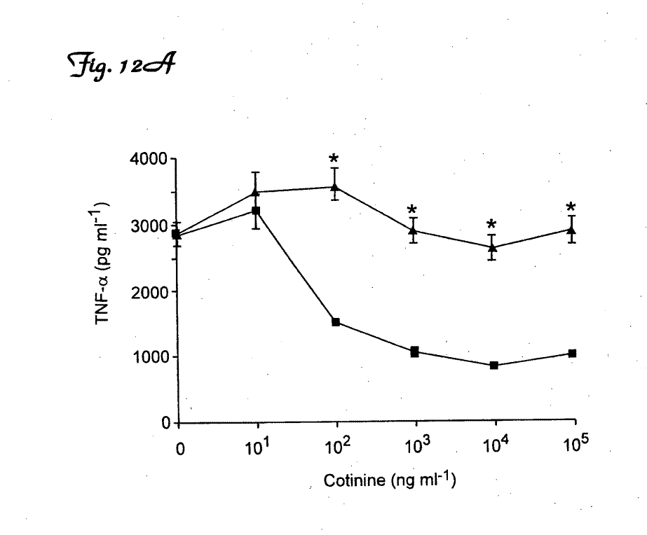

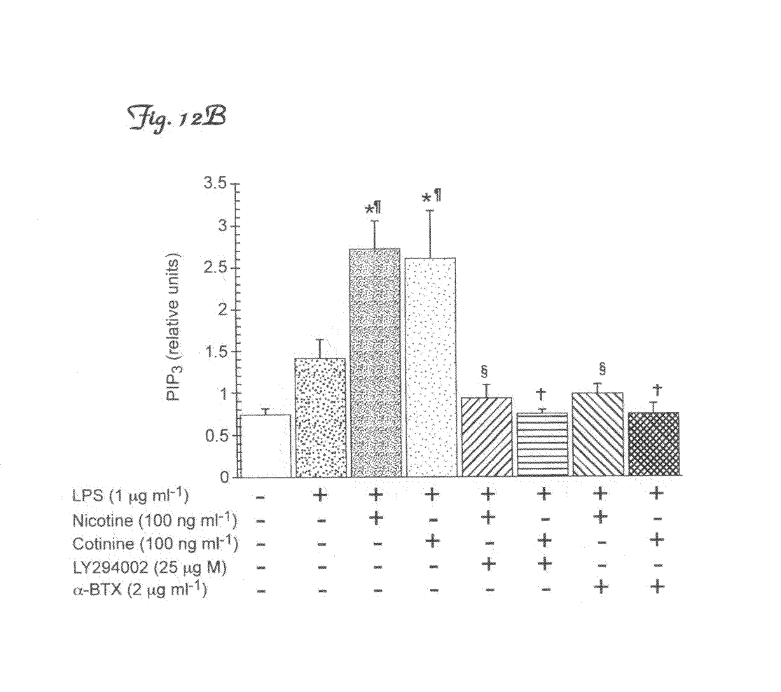

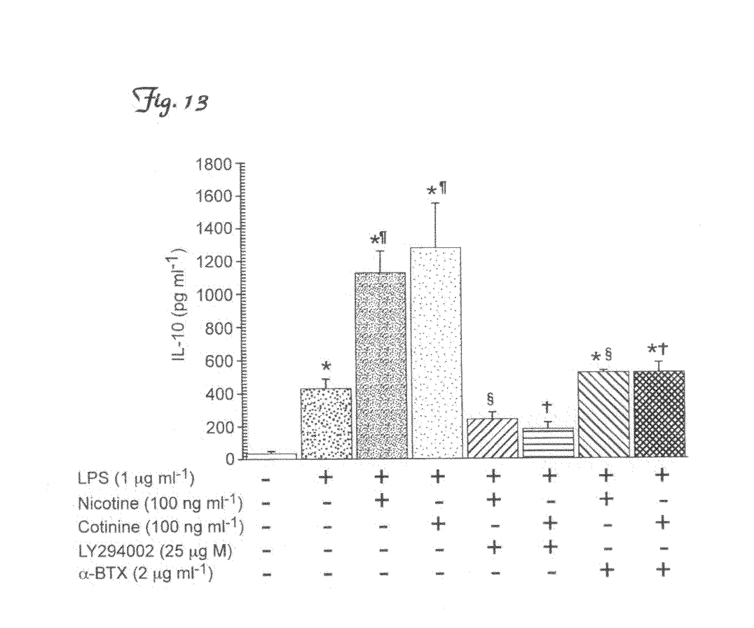

Of the α-bungarotoxin sensitive human nAChRs (α1, α7, and α9), monocytes and macrophages express only functional α7 receptors. The α7 receptor expressed by primary macrophages has a relative molecular mass of 55 kDa by western blot, identical to that of the neuronal receptor. Cotinine-induced suppression of TNF release was abrogated by pre-treatment with α-bungarotoxin (FIG. 10), suggesting that the anti-inflammatory activity of cotinine may be mediated via the nicotinic acetylcholine receptor α7 subunit. The importance of α7 nAChR in cotinine-induced immunomodulation is also suggested by the ability of α-bungarotoxin to block cotinine enhancement of IL-10 release (FIG. 13) and to abrogate cotinine-induced alterations to PI3K activity (measured as PIP₃ release; FIGS. 12A and 12B).

The Anti-Inflammatory Potential of Cotinine is a PI3K/GSK-3β-Dependent but NF-κB-Independent Phenomenon.

Physiologically relevant doses of cotinine augment IL-10 production in LPS-stimulated monocytes, yet LPS-induced NF-κB is not affected by cotinine (FIG. 11). Therefore, cotinine appears to act as an anti-inflammatory agent in an NF-κB-independent manner. It has been previously shown that pharmacological inhibition of GSK-3β enhances IL-10 production in response to LPS. However, SB216763 inhibition of GSK-3β is a significantly less potent inducer of IL-10 than cotinine (FIG. 11). To explore this further, the inventors examined the effects of cotinine on the activation state of Akt (Ser473) and GSK3-β (Ser9). Cotinine enhances levels of phosphorylated (active) Akt in a PI3K-dependent manner and augments levels of phosphorylated (inactive) GSK3-β in P. gingivalis stimulated monocytes.

The Cholinergic and PI3K Anti-Inflammatory Pathways are Convergent on Cotinine Stimulation.

Cotinine potently augments the activation of PI3K and blockade of PI3K abrogates the ability of cotinine to suppress the inflammatory response (TNF-α) while augmenting the production of the anti-inflammatory cytokine IL-10 in P. gingivalis-stimulated cells (FIGS. 12A and 13). Moreover, inhibition of the α7 nAChR-signaling pathway abrogated the ability of cotinine to enhance PI3K activity (FIG. 12B).

Discussion

Electrical stimulation of the vagus nerve protected against death by LPS toxicity in mice. Further, acetylcholine significantly attenuated the release of TNF, IL-β, and IL-6 from macrophages. It is important to note, however, that such vagal protection is abrogated by splenectomy. Specifically, it is the α7 nACh receptor subtype that appears key to acetylcholine-induced inflammatory suppression. α7 nAChR-deficient mice are not only hypersensitive to LPS, producing high amounts of TNF-α, they also exhibit an exaggerated production of the pro-inflammatory cytokines IL-1β and IL-6. Furthermore, nicotine-dependent suppression of TNF release from primary macrophages is abrogated by α7 AChR-specific antisense oligonucleotides, while selective α7 AChR agonists protect against severe sepsis.

Functional AChRs are pentameric and are composed of multiple combinations of a possible 16 monomer subtypes (α1-7; α9-10; β1-4; δ; ε; and γ) that exhibit divergent pharmacological behaviors. nAChR subtype expression by monocytes and macrophages is highly restricted. The α7 receptor expressed by primary macrophages has a relative molecular mass of 55 kDa by western blot, identical to that of the neuronal receptor. Of the known AChRs, α7 nAChR exhibits a number of unusual features. First of all, it can assemble and function as a homopentamer; the ion channel exhibits high permeability for calcium ions in preference to Na⁺; and it is widely expressed in the central and peripheral nervous system as well as on leukocytes. αBTX is not specific for α7 nAChR. However, of the α-bungarotoxin sensitive human nAChRs (α1, α7, and α9), monocytes and macrophages express only functional α7 receptors. The inventors have been able to use α-bungarotoxin to show that cotinine-induced inflammatory suppression occurs through the nicotinic acetylcholine receptor α7 subunit.

The inventors have demonstrated that cotinine has anti-inflammatory properties. They have also shown that cotinine blocks the production of multiple pro-inflammatory cytokines that are known to be under the transcriptional control of NF-κB, including the innate/adaptive immunity bridging cytokine, IL-12/IL-23 p40, while simultaneously augmenting the release of the anti-inflammatory molecule, IL-10. They have also established that these actions of cotinine are likely to be mediated through the α7 nAChR. The suppression of the p40 subunit shared by IL-12 and IL23 is particularly interesting as it is critical for the development of IFN-α-producing Th1 cells and influences cell-mediated immunity and IgG2a antibody production from B cells and, thus, bridges the innate and adaptive immune systems.

The inventors investigated the mechanisms by which cotinine exerts anti-inflammatory effects on innate cells. The NF-κB pathway is critical in the activation of mononuclear phagocytes and in the production of multiple pro-inflammatory cytokines by these cells. Limited evidence suggested that nicotine maintained cytoplasmic concentrations of IκB and thus prevents LPS-induced NF-κB activation in monocytes and macrophages, in a dose-dependent manner. One recent report suggested that smoking inhibits the TLR-2/-4 inducible phosphorylation of IRAK-1 and p38 and also inhibits IκB-α degradation. However, the present data showed that cotinine acted as an anti-inflammatory agent in an NF-κB-independent manner, as physiologically relevant doses of cotinine augment IL-10 production in LPS-stimulated monocytes, yet LPS-induced NF-κB was not affected by cotinine. Indeed, the inventors demonstrated that cotinine regulated the phosphorylation of GSK3β (Serine 9) via the PI3K-Akt pathway.

Once phosphatidylinositol 3 kinase (PI3K) becomes activated, it catalyzes the production of phosphatidylinositol-3,4,5-trisphosphate (PIP3). The generation of PIP3 subsequently allows for the recruitment and co-localization of phosphoinositide-dependent kinase 1 (PDK1) and the serine/threonine-dependent kinase Akt via their pleckstrin homology domains. This co-localization to the plasma membrane allows for the activation of Akt via the phosphorylation of threonine 308 by PDK1 and phosphorylation at serine 473 by a still unidentified kinase called PDK2. Upon dual phosphorylation, Akt becomes activated and phosphorylates a multitude of downstream targets including the serine phosphorylation that results in the inhibition of glycogen synthase kinase 3 (ser21, GSK-3β; and ser9 GSK-3β). Previous studies from the inventors' laboratories have identified that the phosphatidylinositol-3 kinase (PI3K) pathway plays a fundamental role in regulating the host inflammatory response to P. gingivalis by negatively regulating IL-12 p40/p70 production while concurrently augmenting IL-10 levels in a toll like receptor (TLR)-dependent manner. Sequential downstream mapping of the PI3K pathway identified that inactivation (by phosphorylation) of the constitutively active serine/threonine kinase, glycogen synthase kinase 3, via Akt, is central to the ability of this pathway to suppress the levels of pro-inflammatory cytokines while augmenting anti-inflammatory cytokine production (IL-10). Thus, the PI3K pathway can act as a central regulator in modulating the nature (pro vs. anti-inflammatory) and magnitude (absolute levels) of the host inflammatory response. However, at this stage it is important to note that this PI3K anti-inflammatory pathway is normally minimally engaged.

In summary, the inventors have shown that cotinine is a potent anti-inflammatory agent that acts through the α7 nAChR resulting in a convergence of the cholinergic anti-inflammatory pathway and an endogenous PI3K-dependent anti-inflammatory pathway in monocytes. The PI3K-dependent anti-inflammatory pathway in monocytes is normally minimally engaged, but signaling through this PI3K-Akt-GSK-3β route is amplified by cotinine, probably on engagement of α7 nAChR.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

1. A method to treat acute inflammation comprising administering a therapeutically effective amount of cotinine, or a pharmaceutically acceptable salt thereof, to a mammal.

2. A method to treat chronic inflammation, excluding inflammatory bowel disorders (IBD), comprising administering a therapeutically effective amount of cotinine, or a pharmaceutically acceptable salt thereof, to a mammal.

3. A method of modulating activity of a cytokine comprising administering a therapeutically effective amount of cotinine, or a pharmaceutically acceptable salt thereof, to a mammal.

4. The method of claim 3, wherein the activity of the cytokine is inhibited.

5. The method of claim 4, wherein the cytokine is IFN-γ IL-1β, IL-6, TNF-α, IL-12/IL-23.

6. The method of claim 3, wherein the activity of the cytokine is stimulated.

7. The method of claim 6, wherein the cytokine is IL-10.

8. The method of any of claims 1 to 7, wherein the compound is formulated into a tablet or capsule, a transdermal delivery system, a chewing gum, a toothpaste, an interocular insert, an inhaler, or an aqueous solution.

9. The method of any of claims 1 to 8, wherein the compound is formulated as a dosage between about 0.5 mg/kg to 100 mg/kg body weight per day.

10. The method of claims of claim 1 or 2, wherein the inflammation is associated with a peripheral disease.

11. The method of any of claim 1 or 2, wherein the inflammation is a pathogen-induced inflammation.

12. The method of claim 11, wherein the pathogen is a gram negative bacterium.

13. The method of claim 11, wherein the pathogen is Porphyromonas gingivalis.

14. The method of claim 1 or claim 2, wherein the inflammation is associated with anaphylactic shock, arthritis, asthma, cholecystitis, chronic obstructive pulmonary diseases, cystitis, dacryoadenitis, dermatomyositis, dermatitis, endocarditis, endometritis, fasciitis, fibrositis, gastritis, gingivitis, glossitis, mastitis, meningitis, myelitis, myocarditis, nephritis, neuritis, orchitis, osteisis, pancreatitis, parotitis, pericarditis, periodontitis, peritonitis, phlebitis, proctitis, prostatitis, pyelonephritis, rhinitis, sepsis and septic shock, sinusitis, stomatitis, synovitis, tendonitis, tonsillitis, vaginitis, vasculitis, vascular diseases, and/or vulvitis.

15. The use of cotinine, or a pharmaceutically acceptable salt thereof, for the manufacture of a medicament useful for the treatment of acute inflammation in a mammal.

16. The use of cotinine, or a pharmaceutically acceptable salt thereof, for the manufacture of a medicament useful for the treatment of chronic inflammation, excluding inflammatory bowel disorders (IBD), in a mammal.

17. The use of cotinine, or a pharmaceutically acceptable salt thereof, for the manufacture of a medicament useful for the modulation of activity of a cytokine in a mammal.

18. A pharmaceutical composition comprising cotinine, or a pharmaceutically acceptable salt thereof, in combination with any steroidal or non-steroidal anti-inflammatory drugs(s), and a pharmaceutically acceptable diluent or carrier.

19. A method to suppress a pro-inflammatory response of monocytes
comprising administering a therapeutically effective amount of cotinine to a mammal.

20. A method to prevent or treat acute bacterial and viral infections
comprising administering a therapeutically effective amount of cotinine to a mammal.

21. A method preventing or treat a TLR-mediated inflammatory condition
comprising administering a therapeutically effective amount of cotinine to a mammal.

22. The method of claim 21, wherein the TLR-mediated inflammatory condition is arthritis, bronchitis, cancers, or vascular inflammation.

23. An article of manufacture comprising, a packaging material and at least one unit dosage form of a pharmaceutical agent contained in the package wherein the pharmaceutical agent comprises cotinine or a pharmaceutically acceptable salt thereof in an amount effective to alleviate or reduce the symptoms of acute and/or chronic inflammation, and wherein the package includes instructions indicating use for alleviating acute and/or chronic inflammation.

24. The article of manufacture of claim 23, wherein the instructions comprise a printed label, printed package insert, tag, cassette tape and the like.

25. The article of manufacture of claim 23, wherein the packaging comprises a box, bottle, tube, spray or insufflator, intravenous bag, envelope or the like.

26. Use of cotinine or a pharmaceutically acceptable salt thereof, in the manufacture of a medicament for the treatment of disease anaphylactic shock, arthritis, asthma, cholecystitis, chronic obstructive pulmonary diseases, cystitis, dacryoadenitis, dermatomyositis, dermatitis, endocarditis, endometritis, fasciitis, fibrositis, gastritis, gingivitis, glossitis, mastitis, meningitis, myelitis, myocarditis, nephritis, neuritis, orchitis, osteisis, pancreatitis, parotitis, pericarditis, periodontitis, peritonitis, phlebitis, proctitis, prostatitis, pyelonephritis, rhinitis, sepsis and septic shock, sinusitis, stomatitis, synovitis, tendonitis, tonsillitis, vaginitis, vasculitis, vascular diseases, and/or vulvitis.
